(19) United States
(12) Patent Application Publication
(10) Pub. No.: US 2015/0211063 A1
(43) Pub. Date: Jul. 30, 2015

(54) Methods of Screening for Susceptibility to Virus Infection

(71) Applicant: THE HENRY M. JACKSON FOUNDATION FOR THE ADVANCEMENT OF MILITARY MEDICINE, INC., Bethesda, MD (US)

(72) Inventors: Haitao Hu, Gaithersburg, MD (US); Silvia R. Kim, Bethesda, MD (US)

(21) Appl. No.: 14/427,595

(22) PCT Filed: Sep. 11, 2013
(86) PCT No.: PCT/US13/59153

§ 371 Date: Mar. 11, 2015

Related U.S. Application Data
(60) Provisional application No. 61/699,456, filed on Sep. 11, 2012.

(51) Int. Cl. C12Q 1/68; C12Q 1/70; G01N 33/50

(52) U.S. Cl. CPC C12Q 1/6881; G01N 33/505; C12Q 1/703; C12Q 2600/136; C12Q 2600/158

(57) ABSTRACT

The invention is directed to methods for screening the resistance of CD4+ cells to viral infection, in particular human immunodeficiency virus (HIV) infection.

### STATEMENT REGARDING FEDERALLY SPONSORED RESEARCH OR DEVELOPMENT

Part of the work performed during development of this invention utilized U.S. Government funds from the United States Department of Defense Grant No. W81XWH-07-2-0067. The U.S. Government has certain rights in this invention.

### REFERENCE TO SEQUENCE LISTING

To be completed.

### BACKGROUND OF THE INVENTION

1. Field of the Invention

The invention is directed to methods for screening the resistance of antigen-specific CD4+ cells to viral infection, in particular human immunodeficiency virus (HIV) infection.

2. Background of the Invention

CD4+ T cells, as a crucial part of immune system, are targets by HIV for infection. Induction of a functional subset of antigen-specific CD4+ T cells that are resistant to HIV infection is highly desirable for HIV vaccine research. Further identification of cellular parameters at the molecular level that are associated with HIV-resistant phenotype of CD4 population is scientifically as well as commercially significant as it provides information on novel targets and candidate genes for anti-HIV drug discovery.

### SUMMARY OF THE INVENTION

The invention is directed to methods for screening the resistance of CD4+ cells to viral infection, in particular human immunodeficiency virus (HIV) infection.

The present invention is also directed to methods of inducing resistance of CD4+ cells to virus infection, the method comprising increasing the transcription of at least one gene selected from the group consisting of IFI44L, IFIT1, IFI27, IFI44, IFIT3, IFI6, OASL, OAS1, OAS2, OAS3, PKR, MDA5, RSAD2, MX1, TRIM22, TRIM5, NKG7, DDX60, IRF7, MX2, IFITM1, ISG20 and DDX58.

### BRIEF DESCRIPTION OF THE DRAWINGS

FIG. 1 depicts in vitro HIV infection of pathogen-specific CD4+ T cells in peripheral blood mononuclear cells (PBMC). Representative flow cytometric plots are shown for intracellular p24 expression in CFSE-low population of CD3+CD8+ T cells in antigen-stimulated PBMC with (top) or without (bottom) infection by R5-HIV (A) or X4-HIV (C). Only live CD14-19-CD3+CD8− T cells were gated for analysis. The data were expressed as proportion of intracellular p24+ cells in CFSE-low population. Comparison of proportion of p24+CFSE-low cells between CMV-, TT- and Candida-specific CD4+ T cells from multiple subjects is shown as box and Whisker plots in B (R5 HIV) and D (X4 HIV). Relative quantification of infectious R5 HIV (E) or X4 HIV (F) viruses produced by pathogen-specific CD4+ T cells in supernatants. Quantification was based on infection of TZM-bl cells and the data expressed as RLU. Statistical analysis was performed using the Mann-Whitney test. ***p<0.005; **p<0.01, *p<0.05.

FIG. 2 depicts the quantification of cell-associated HIV DNA in pathogen-specific CD4+ T cells after infection. (A) Pathogen-specific CD4+ T cells were sorted from PBMC by FACS Aria based on CFSE_{-low}. (B) Quantification of cell-associated strong-stop and full-length HIV DNA in sorted pathogen-specific CD4+ T cells. The results were expressed as fold increase in HIV DNA copies for TT- and Candida-specific CD4+ T cells relative to CMV-specific CD4+ T cells (mean and SD). Statistical analysis was performed using the Mann-Whitney test. *p<0.05

FIG. 3 depicts the effect of β-chemokine neutralization on cell-associated HIV DNA and p24 contents in pathogen-specific CD4+ T cells. (A) Expression of CCR5 on pathogen-specific CD4+ T cells, expressed as proportion (left) or intensity (right), is shown. (B) Effect of β-chemokine neutralization on cell-associated HIV gag DNA content in pathogen-specific CD4+ T cells. Pathogen-specific CD4+ T cells with or without treatment by neutralization antibodies (anti-MIP-1α, anti-MIP-1β, and anti-RANTES were subject to real-time PCR for quantification of cell-associated HIV full-length DNA. The results were expressed as fold change in HIV DNA copies for cells with neutralization relative to no neutralization treatment within each pathogen-specificity. (C) Effect of β-chemokine neutralization on intracellular p24 content. CFSE-loaded PBMC were antigen-stimulated and HIV infected in the absence or presence of individual anti-β-chemokine antibodies alone or in combination. Cells were subject to p24 flow cytometric analysis. The results were expressed as proportion of p24+CFSE_{-low} cells in each group of pathogen-specific CD4+ T cells. Statistical analysis was performed using the Mann-Whitney test. ***p<0.005; **p<0.01, *p<0.05

FIG. 4 depicts the transcriptomic analysis of pathogen-specific CD4+ T cells. (A) Global view of fold changes in gene expression for genes that were significantly (FDR q-value<0.05) upregulated in CMV-specific CD4+ T cells (667, top points) or in TT- and Candida-specific CD4+ T cells (1171, bottom points). (B) A heat map for global comparison of gene expression changes between CMV-specific (middle) and TT-specific (right) or Candida-specific-(left) CD4+ T cells from three subjects. Relative upregulation and downregulation of mRNA levels are shown. (C) Functional category and gene ontology enrichment analysis using DAVID based on significant genes identified by SAM (first three bars: CMV, last three bars: TT-candida). The number of significant genes and p-value for each category was shown. (D) List of genes that are upregulated in CMV-specific CD4+ T cells and associated with antiviral responses. The fold increase for each gene is shown. (E and F) List of genes that are upregulated in TT-specific (E) or Candida-specific (F) CD4+ T cells and associated with Th17 and inflammatory responses. Data are shown as geometric means of fold increases for three subjects. *FDR q-value<0.05; N.S.: no significance

### DETAILED DESCRIPTION OF THE INVENTION

The invention is directed to methods for screening the resistance of CD4+ cells to viral infection, in particular human immunodeficiency virus (HIV) infection. In general, the methods of screening resistance of CD4+ cells comprise labeling CD4+ cells with carboxyfluorescein diacetate, succinimidyl ester (CFSE) or carboxyfluorescein diacetate, succinimidyl ester (CFDA-SE).

Method of labeling the CD4+ cells with CFSE are well known in the art. CFSE is commercially available, and one of skill in the art can routinely follow the manufacturer's recommended instructions for labeling the cells as desired.

The cells used in the methods of the present invention are CD4+ cells. In one embodiment, peripheral blood mononuclear cells (PBMC) contain the CD4+ cells. In a more specific embodiment, the CD4+ cells are isolated from the PBMC fraction prior to carrying out any of the methods of the present invention. In another specific embodiment, the CD4+ cells are not isolated from the PBMC fraction prior to carrying out any of the methods of the present invention. In more specific embodiments, the CD4+ cells used in the methods of the present invention, whether isolated from PBMCs or not, comprise naïve CD4+ cells. In another embodiment, the CD4+ cells used in the methods of the present invention, whether isolated from PBMCs or not, comprise memory CD4+ cells. In another embodiment, the CD4+ cells used in the methods of the present invention, whether isolated from PBMCs or not, comprise regulatory CD4+ cells. In yet another embodiment, the CD4+ cells used in the methods of the present invention, whether isolated from PBMCs or not, comprise a mixture of one or more populations of naïve CD4+ cells, memory CD4+ cells and regulatory CD4+ cells.

The term “CD4+ cells” is well understood in the art and is used to mean cells that express the CD4 cell surface marker. One of skill in the art will readily understand that CD4+ cells are T helper cells that occur in an animal with an adaptive immune system. As used herein, “isolated CD4+ cells” is used to indicate that the CD4+ cells are isolated from PBMCs. Of course, it is understood that PBMCs are a portion of whole blood. Thus, in one embodiment the methods of the invention comprise using whole blood comprising PBMCs. In another embodiment, the methods of the invention comprise fractionating PBMCs from whole blood prior to subjecting it to the methods of the present invention. In yet another embodiment, the methods comprise isolating CD4+ cells, either from whole blood, the PBMC fraction or another CD4+ cell-containing fraction of whole blood prior subjecting the cells to the methods of the present invention. The term “blood” is used herein as shorthand to include and encompass the terms and concepts of whole blood, the PBMC fraction and any other CD4+ cell-containing fraction of whole blood, including but not limited to isolated CD4+ cells.

Methods of fractionating whole blood to obtain PBMCs are well known in the art. In addition, methods of isolating CD4+ cells from whole blood, PBMCs or another CD4+ cell-containing fraction of whole blood are also well known. The blood used in the methods of the present can be from any source, including but not limited to a freshly drawn blood from an animal, such as a human, or from cryopreserved storage facilities. In one embodiment the blood used in the methods of the present invention are from an individual that has not been infected with the subject virus that is being investigated in the methods of the present invention. For example, if HIV, SIV or SHIV infectivity is being investigated, the blood used in the methods of the present invention would be from an individual that has not been infected with HIV, SIV or SHIV.

The methods comprise contacting the labeled CD4+ cells with at least one stimulating composition. The stimulating compositions with which the CD4+ cells are contacted can be any stimulating composition that is known to or suspected of being able to stimulate activation and proliferation of memory CD4+ cells specific to certain antigens. The stimulating composition used in the methods of the present invention may comprise a small molecule, such as an organic pharmaceutical compound or any other “non-biologic” compound. A biological compound or “biologic” is understood in the art to comprise a compound comprising amino aids and/or nucleic acids, thus a “non-biologic” would be understood to encompass molecules not comprising amino acids or nucleic acids. The stimulating composition used in the methods of the present invention may also comprise a biologic compound. In one embodiment, the stimulating compositions used in the methods of the present invention comprise both small molecules and biologics.

Examples of biologics comprise but are not limited to proteins, peptides, nucleic acids and the like. As used herein, a biologic includes vaccines, potential vaccines and antigenic portions of proteins, polypeptides and/or pathogens. As is readily understood, a vaccine generally includes attenuated whole organisms or portions thereof, proteins or other components that generally include an antigen capable of stimulating a recipient's immune system. In one embodiment, the compositions used in the methods of the present invention comprise one or more antigens, such as 2, 3, 4, 5, 6, 7, 8, 9 or more antigens.

In specific embodiments, the stimulating compositions used in the methods of the present invention comprise at least one antigen that is or is derived from an animal pathogen. Examples of pathogens are well known and include but are not limited to the general category of organisms selected from a virus, a bacterium, a prion, a fungus, a protozoan and an animal (such as nematode, helminths or other worm). The stimulating compositions need only contain an antigen derived from the pathogen, but, of course, the compositions used in the present invention may comprise the entire organism. One of skill in the art will readily understand how to derive an antigen from a pathogen if such a composition is desired. An antigen that is “derived from a pathogen” includes but is not limited to isolated portions of the pathogen that elicit an antigenic response, such as a surface protein or portion thereof, toxins generated from the pathogens, toxoids and the like. Indeed, many antigenic determinants of a variety of pathogens are well known in the art. It is not, however, necessary that the identity of the antigen, e.g., an amino acid sequence, for carrying out the methods of the present invention. In fact, in one embodiment, the methods of the present of the invention are not dependent on the identity of the antigen. For example, the stimulating composition that is placed into contact with the CD4+ cells may comprise a cytomegalovirus (CMV), a tetanus toxoid, or the fungus Candida albicans, and it is not necessary that one knows the antigenic portion of these components of the stimulating compositions to perform the methods of the present invention.

Of course, vaccines and any other stimulating composition used in the method of the present invention may or may not include other components such as adjuvants, carriers, vehicles, solvents and the like.

In one embodiment of the present invention, the labeled CD4+ are contacted with the subject virus that is being studied after they have been stimulated by contact with the stimulating composition. In another embodiment, the labeled CD4+ are contacted with the subject virus that is being studied at the same time or roughly the same time that the cells are stimulated by contact with the stimulating composition. As used herein, the term “subject virus” is the virus being studied for its infectivity on the CD4+ cells. In general, the CD4+ cells should be from an individual organism that has not been infected with the subject virus.

Methods of contacting the CD4+ cells with a subject virus are well known in the art. In general, a virus preparation comprises supernatant from a cell culture in which the cells were infected with the subject virus. The supernatant is then applied to the CD4+ cells being studied. If a supernatant is being used to contact the subject virus to the CD4+ cells, the supernatant may or may not be processed prior to its application. Such additional processing may or may not include filtration, centrifugation, dialysis and the like.

Other methods of contacting the CD4+ cells with the subject virus include but are not limited to transfection of the subject virus into the CD4+ cells such that the subject virus's DNA or RNA is inserted directly into the cells. Transfection methods are well known in the art. The method of contacting the subject virus to with the CD4+ cells is not a limiting factor in the invention, and any methods of contact the CD4+ cells with the subject virus will suffice, provided the subject virus is able to infect control CD4+ cells.

In one embodiment of the present invention, a test substance is also applied to the CD4+ cells. The test substance should be different from the stimulating composition. In this embodiment, a test substance is applied to the CD4+ cells either before, during or after application of the subject virus. The infectivity of the subject virus in response to the test substance can then be monitored. The response of the cells to the test substance is monitored to determine if the test substance alters the ability of the subject virus to infect the CD4+ cells. The test substance may enhance, decrease or not alter the ability of the subject virus to infect the CD4+ cells. In this manner, the invention provides for methods of screening test substances as potential therapeutics or prophylactics of virus infection. The parameters of the methods can be altered to accommodate different subject viruses and different test substances as desired.

After application of the subject virus to the CD4+ cells, virus infectivity is then assessed in the CD4+ cells. In one embodiment, proliferation rates of the CD4+ cells are monitored as an assessment of magnitude of memory CD4 response to given antigens and as way to identify antigen-specific CD4 T cell populations out of bulk PBMC. Any method of assessing proliferation rates may be employed in the methods of the present invention, and the inventive methods are not dependent on the ways of assessing proliferation rates of the CD4+ cells that have been contacted with the subject virus. In one specific embodiment, fluorescence activated cell sorting (FACS) technology is used to monitor or assess proliferation rates of the CD4+ cells that have been contacted with the subject virus.

In another embodiment, the appearance or prevalence of virus markers is monitored in the CD4+ cells as a way of assessing virus infectivity. In general, an increase in the levels of the subject virus marker are indicative that the cells are susceptible to infection of the subject virus, whereas reduced or undetected levels of the marker would be indicative that the cells are at least partially resistant to infection of the subject virus. For example, if HIV infectivity is being assessed, the appearance or prevalence of the p24 protein, which is a well-known marker for HIV infection, in the CD4+ cells can be determined as a way of assessing virus infectivity. Continuing the example, if CD4+ cells demonstrate an increase in the p24 marker after being contacted with HIV, it would be understood that these cells are more susceptible to HIV infection compared to cells that exhibit reduced levels of p24 marker. Any method of assessing levels of subject virus markers can be employed in the methods of the present invention. In one specific embodiment, FACS technology is used to monitor or assess the appearance or prevalence of the selected markers of the subject virus.

A lower viral infectivity in the stimulated, labeled CD4+ cells compared to control cells would indicate that the stimulated, labeled CD4+ cells are at least partially resistant to infection of the subject virus. As used herein, the term “control cells” is well understood and is used to mean the cells that are not subjected to the test variable. For example, control cells may be cells on which the subject virus has not been applied. In this manner, the infectivity of the virus is assessed by comparing the proliferation rates or virus marker prevalence in cells contacted with the subject virus to cells that have not been contacted with the subject virus. In another example, the control cells may be cells on which a test substance has not been applied. The control cells in this embodiment would be cells on which the subject virus has been applied, but on which the test substance has not been applied. Similarly, control cells would also be cells on which neither the test substance nor the subject virus has been applied.

Comparing the response of the test cells to the control cells can comprise any method that will highlight any differences in cell populations if they exist. The comparison can be qualitative or quantitative. Furthermore, the quantitative differences can be relative or absolute. Of course, the differences in proliferation or marker prevalence compared to control levels may be equal to zero, indicating the that the treated cells are as susceptible to infection control cells. The quantity may simply be the measured rates or marker levels without any additional measurements or manipulations. Alternatively, the differences in rates or levels may be manipulated mathematically or in an algorithm, with the algorithm designed to correlate the measured rates or marker levels to the ability of the virus to infect the CD4+ cells. If, for example, a test substance is being used, the quantity may be expressed as a difference, percentage or ratio of the measured value of the rates or markers to a value or values of another substance including, but not limited to, a standard. The differences may be negative, indicating that the CD4+ cells are at least partially resistant to infection compared to control cells, or the differences may be positive, indicating that the CD4+ cells are at least partially susceptible to infection compared to control cells. Of course, any algorithm or mathematical manipulation of the data may reverse the sign (negative or positive) of the data.

The quantity may also be expressed as a difference or ratio of rates or markers measured at different points in time to assess the progression of infectivity in response to a test substance. Thus the invention provides for methods of monitoring the progression of infectivity or monitoring the ability of a test substance to affect virus infectivity over time by performing the methods described herein over multiple time points and comparing the data over time.

The methods of screening the susceptibility of CD4+ cells to infection from a subject virus can optionally include determining the transcription or transcription rates of at least one gene in the stimulated, labeled CD4+ cells. The CD4+ cells in which transcription or transcription rates are assessed may be cells demonstrating an increased or decreased resistance to infection of the subject virus compared to control cells. Transcription or transcription rates of the at least one gene may be increased or decreased in the CD4+ cells being studied over controls, i.e., CD4+ cells that are either more or less resistance to infection viral infection.

Methods of assessing transcription and transcription rates in cells are well known in the art and are routine to one of skill in the art. For example, reverse transcription (RT) polymerase chain reaction (PCR) can be used to assess the presence or absence of transcription in cells, quantitative PCR can be used to assess levels of transcription, real-time PCR can also be used to assess transcription and transcription rates. Nuclear run-on assays can also be used to assess rates and timing of transcription of genes within a given population of cells. In addition, methods and procedures that measure protein products can be used as an indirect assessment of transcription or transcription rates. For example, enzyme-linked immunosorbent assays (ELISA) and Western Blot analysis can be used to measure protein products as a result of increased transcription. In addition, FACS can also be used to assess protein products in the CD4+ cells as an indirect measurement of transcription or transcription rates. Transcription rates can be approximated, for example, by comparing transcription products (RNA or protein) at different points in time, or transcription rates can be assessed directly from the assay itself, e.g., real-time PCR.

The transcription or transcription rate of any gene can be assessed. Examples of categories of genes whose transcription or transcription rates that might be assessed include but are not limited to interferon-inducible (IFI) genes, anti-viral RNA responsive genes, anti-viral defensive genes, virus restriction factors to name a few.

Specific examples of genes whose transcription or transcription rates might be assessed in some of the embodiments of the present invention include but are not limited to the genes or the genes encoding interferon-induced protein 44-like (IFI44L), interferon-induced protein with tetratricopeptide repeats 1 (IFIT1), interferon alpha-induced protein 27 (IFI27), interferon-induced protein 44 (IFI44), interferon-induced protein with tetratricopeptide repeats (IFIT3), interferon alpha-induced protein 6 (IFI6), 59 kDA 2′-5′-oligoadenylate synthase-like protein (OASL) (also known as thyroid receptor-interacting protein 14), 2′-5′-oligoadenylate synthase 1 (OAS1), 2′-5′-oligoadenylate synthase 2 (OAS2), 2′-5′-oligoadenylate synthase 3 (OAS3), interferon-induced, double-stranded RNA-activated protein kinase (PKR), interferon-induced helicase C domain-containing protein 1 (MDA5), radical S-adenosyl methionine domain-containing protein 2 (RSAD2), interferon-induced GTP-binding protein Mx1 (MX1), E3 ubiquitin-protein ligase TRIM22 (TRIM22), protein TRIM5 (TRIM5), protein NKG7 (NKG7), probable ATP-dependent RNA helicase DDX60 (DDX60), interferon regulatory factor 7 (IRF7), interferon-induced GTP-binding protein Mx2 (MX2), interferon-induced transmembrane protein 1 (IFITM1), interferon stimulated gene 20 kDa protein (ISG20) and probable ATP-dependent RNA helicase DDX58 (DDX58). Transcription or transcription rates of any number of these genes or genes encoding the proteins listed above can assessed. In select example, transcription or transcription rates are assessed in a number of a number of genes selected from the group consisting of 1, 2, 3, 4, 5, 6, 7, 8, 9, 10, 11, 12, 13, 14, 15, 16, 17, 18, 19, 20, 21, 22, 23, 24 and 25. In one specific embodiment transcription or transcription rates are assessed for one, two, three of four of IFI27, IFI44, IFI44L and/or IFIT1.

Assessing transcription or transcription rates of genes in cells that demonstrate at least partial resistance to virus infection can be useful to focus research on specific genes that could be useful in staving off infection in subjects that are susceptible to infection from the virus. In the alternative, assessing transcription or transcription rates of genes in cells that demonstrate at least partial susceptibility to virus infection can be useful to focus research on specific endogenous genes that viruses utilize during the infection process.

The methods of the present invention can be applied to virtually any subject virus that can infect CD4+ cells. Examples of subject viruses that might be studies using the methods of the present invention include but are not limited to human immunodeficiency virus (HIV), simian immunodeficiency virus (SIV), simian human immunodeficiency virus (SHIV) and cytomegalovirus (CMV).

The present invention also provides methods of inducing resistance of CD4+ cells to virus infection, for example HIV infection, with the methods comprising increasing the transcription IFI44L, IFIT1, IFI27, IFI44, IFIT3, IFI6, OASL, OAS1, OAS2, OAS3, PKR, MDA5, RSAD2, MX1, TRIM22, TRIMS, NKG7, DDX60, IRF7, MX2, IFITM1, ISG20 or DDX58. The methods may also comprise increasing transcription of more than one gene or gene encoding the proteins listed above. In specific embodiments, the methods comprise increasing transcription of 2, 3, 4, 5, 6, 7, 8, 9, 10, 11, 12, 13, 14, 15, 16, 17, 18, 19, 20, 21, 22 or 23 of genes from the genes or proteins encoded by the genes list above.

Methods of increasing transcription include but are not limited to transfecting cells with nucleic acids encoding at least one additional copy of the genes or gene products listed above. Methods of transfecting cells are established and well known in the art. The additional copies of the nucleic aids may or may not be from the same organism. For example, nucleic acids of one or more of the genes listed herein may encode the mouse version of a particular gene, and this nucleic acid may be transfected into CD4+ cells of a different organism, e.g., human CD4+ cells.

The methods of increasing the transcription of at least one gene may also comprise administering an agent to the CD4+ cells that cause transcription of the at least one gene. The agent can be any agent that causes an increase in transcription of the one or more genes, including but not limited to the stimulating compositions described herein.

The invention also provides methods of screening a test substance for its ability to increase resistance of CD4+ cells to virus infection, for example HIV infection. The screening methods comprise contacting CD4+ cells with the test substance and assessing transcription levels of at least one gene selected from the group consisting of IFI44L, IFIT1, IFI27, IFI44, IFIT3, IFI6, OASL, OAS1, OAS2, OAS3, PKR, MDA5, RSAD2, MX1, TRIM22, TRIM5, NKG7, DDX60, IRF7, MX2, IFITM1, ISG20 and DDX58 as described herein. An increase in transcription levels of the at least one of the genes compared to control levels indicates that the test substance will at least partially increase the resistance of CD4+ cells to viral infection.

In select embodiment, the methods comprise assessing transcription of number of genes selected from the group consisting of 1, 2, 3, 4, 5, 6, 7, 8, 9, 10, 11, 12, 13, 14, 15, 16, 17, 18, 19, 20, 21, 22 and 23. In one specific embodiment, the methods comprise assessing transcription or transcription rates of one or more of IFI27, IFI44, IFI44L and/or IFIT1.

The test substance used in these methods can be any test substance described herein and includes, but is not limited to a small molecule, a biologic, including but not limited to vaccines.

The examples disclosed herein are meant to be illustrative in nature and are not intended to limit the scope of the invention in any way.

### EXAMPLES

Peripheral blood mononuclear cells (PBMC) samples were obtained from HIV negative subjects enrolled in an IRB-approved protocol, RV229, at Walter Reed Army Institute of Research (WRAIR). Cryopreserved PBMC were thawed and maintained in complete RPMI 1640 (Invitrogen) supplemented with 10% human serum, 100 U/ml penicillin G, 100 U/ml streptomycin sulfate, and 1.7 mm sodium glutamine. PBMC with positive responses to cytomegalovirus (CMV), tetanus toxoid, and Candida albicans, were chosen for this study (n=6). R5 tropic HIV (US1) and X4 tropic HIV viruses (92/UG/029) were obtained from the Department of Vaccine Research and Development at US Military HIV Research Program (MHRP). Antigens for pathogen-specific stimulation of PBMC include CMV viral lysates (Advanced Biotechnologies, Inc.) or pp65 peptide pools (JPT, Peptide Technology), tetanus toxoid (Statens Serum Institut) and Candida albicans sonicate (Greer Labs).

PBMC were labeled with carboxyfluorescein diacetate, succinimidyl ester (CFSE) as previously described with slight modification (Hu, H., Fernando, K., Ni, H. & Weissman, D. HIV envelope suppresses CD4+ T cell activation independent of T regulatory cells. J Immunol 180, 5593-5600 (2008)). In brief, 30×10⁶ PBMC were washed and re-suspended in 1 ml staining media (RPMI 1640 with 1% NHS) containing 1 μM CFSE. Staining was performed at 25° C. for 8 minutes. Cells were then mixed with 2 ml pre-warmed 100% NHS for 5 minutes to quench the CFSE. CFSE-labeled PBMC were equally divided and pulsed with antigens (CMV: 5 μg/ml; tetanus toxoid: 25 μg/ml; Candida albicans: 1:200) at concentration of about 10×10⁶ cells/ml 4 hours, and then diluted to concentration of 2×10⁶ cells/ml for continuous culture for 3 days to allow for antigen-specific proliferation. Unstimulated and 1 μg/ml SEB-stimulated (Sigma) PBMC were included as controls. After stimulation, cells were infected with pre-titrated R5 HIV (US1) or X4 HIV (92/UG/029). 24 hours after infection, cells were washed to remove uninfected free HIV. Cells were subject to flow cytometric p24 analysis 3 days after infection. In some experiments, anti-MIP-1α (5 μg/ml; Clone: 93321; R&D system), anti-MIP-1β (5 μg/ml; Clone: 24006; R&D system) or anti-RANTES (5 μg/ml; Clone: 21418; R&D system) was added into culture alone or in combination during antigen stimulation and HIV infection.

TZM-bl cells were plated at the concentration of about 5×10⁴ cells/ml. 12 hours later, supernatants containing HIV virus were added at 1:2 serial dilutions in the presence of 40 μg/ml of DEAE-dextran hydrochloride (Sigma). TZM-bl cells are a HeLa cell clone line that are engineered to express CD4 and CCR5 and contain integrated reporter genes for firefly luciferase and E. coli β-galactosidase under control of an HIV-1 long terminal repeat (LTR), permitting sensitive and accurate measurements of infection. Virus infectivity was determined 48 hours post-inoculation by measuring the level of Firefly luciferase activity expressed in infected cells (Bright-Glo™, Promega). Each experiment was performed in duplicate.

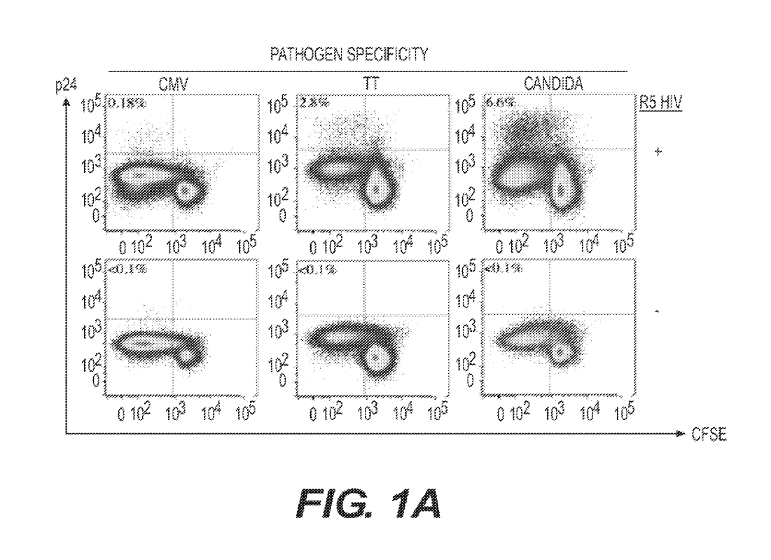

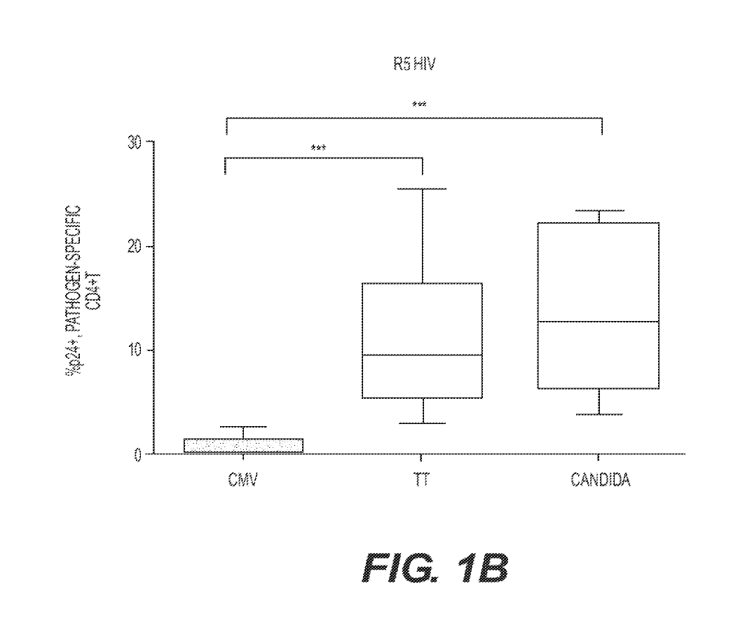

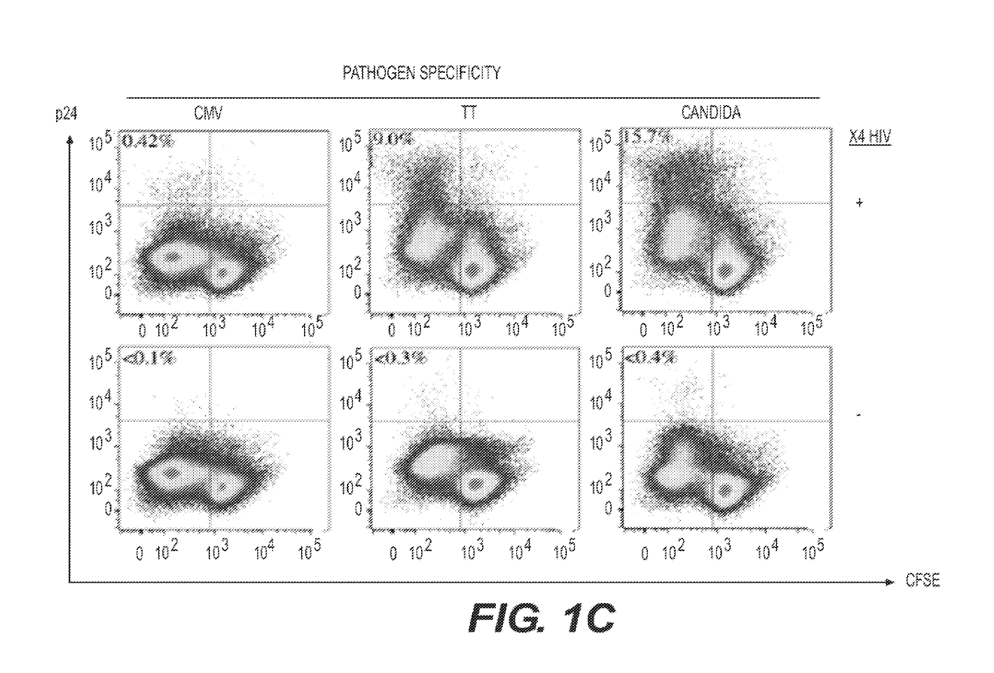

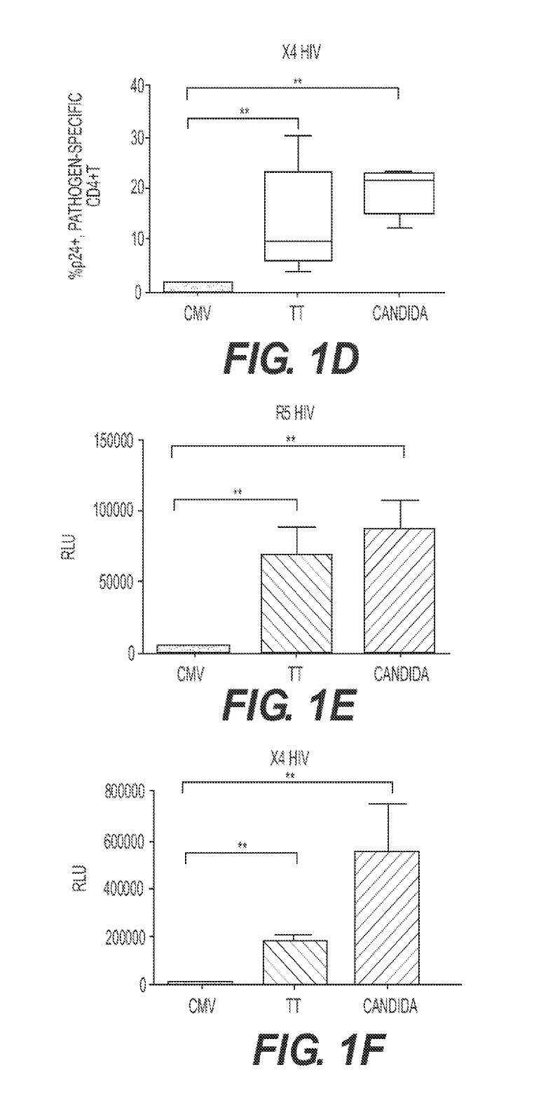

After infection with R5 tropic HIV, TT- and Candida-specific CD4+ T cells exhibited p24+ rate of 2.8% and 6.6% respectively, whereas only 0.18% of CMV-specific CD4+ T cells expressed p24 (FIG. 1A). The difference in R5-HIV infection between pathogen specific CD4+ T cells was statistically significant (p<0.005 for CMV vs. TT and CMV vs. Candida) (FIG. 1B). Similar results were observed when cells were infected with X4 tropic HIV (CMV: 0.42%; TT: 9.0%; Candida: 15.7%) (p<0.01 for CMV vs. TT and CMV vs. Candida) (FIGS. 1C and 1D).

The amount of infectious HIV particles produced according to TZM-bl infection by supernatants was also determined, and significantly more HIV was produced in TT- and Candida-stimulated than CMV-stimulated PBMC for both R5 (FIG. 1E) and X4 (FIG. 1F) HIV infection. These results showed that while TT- and Candida-specific CD4+ T cells were permissive and produced de novo functional HIV particles, CMV-specific CD4+ T cells were highly resistant to both X4 and R5 HIV infection independent of viral tropism.

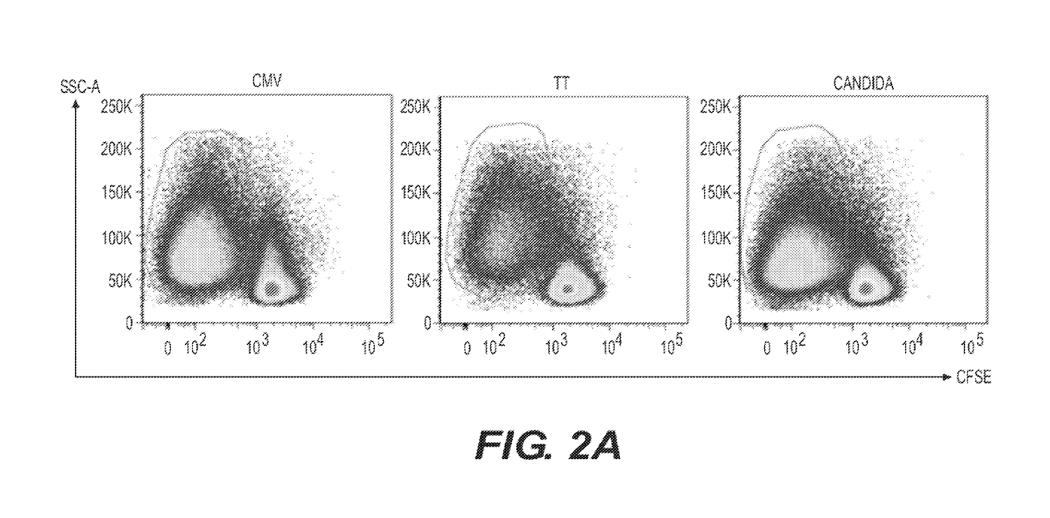

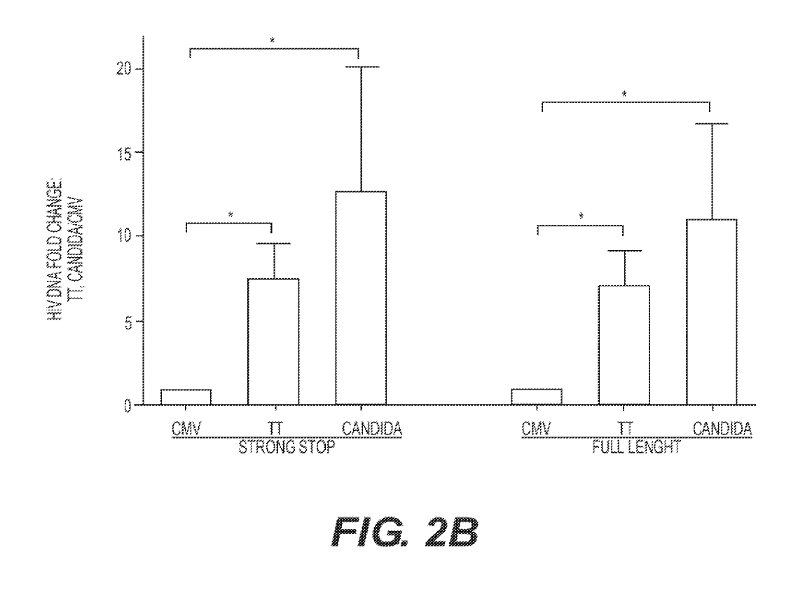

Analysis of p24 in the supernatants showed results consistent with intracellular p24, with no evidence of spreading infection. Similar to the intracellular p24 results, CMV-specific CD4+ T cells demonstrated a significant reduction in the amounts of both strong-stop and full-length HIV DNA compared to TT- or Candida-specific CD4+ T cells (FIG. 2B). The strong-stop/full-length HIV DNA ratio was comparable between pathogen-specific CD4+ T cells (FIG. 2B), suggesting that HIV reverse transcription is not preferentially impaired in CMV-specific cells. Baseline HIV DNA content remained lower in CMV-specific cells compared to TT- and Candida-specific cells despite β-chemokine neutralization (FIG. 2B, 2C), suggesting that there are possibly other factors associated with CMV specificity that can inhibit HIV entry or early stages before reverse transcription.

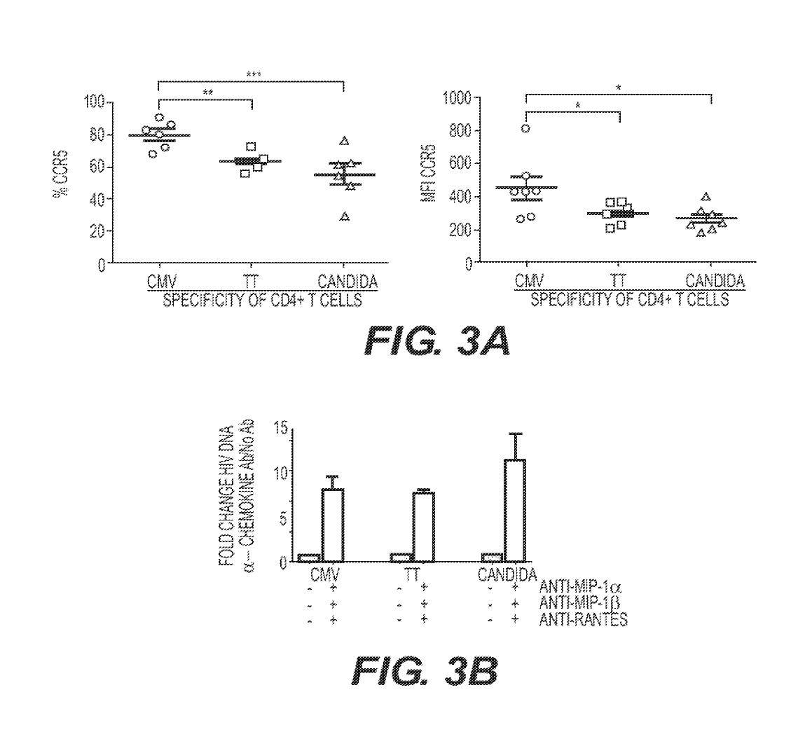

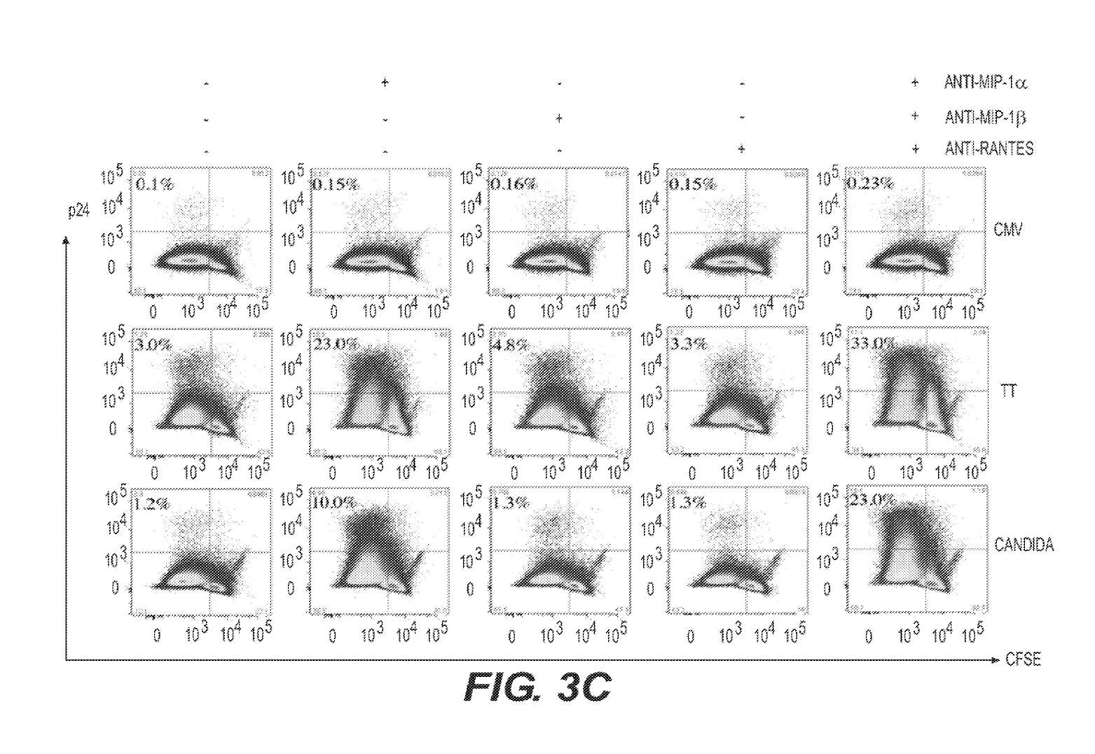

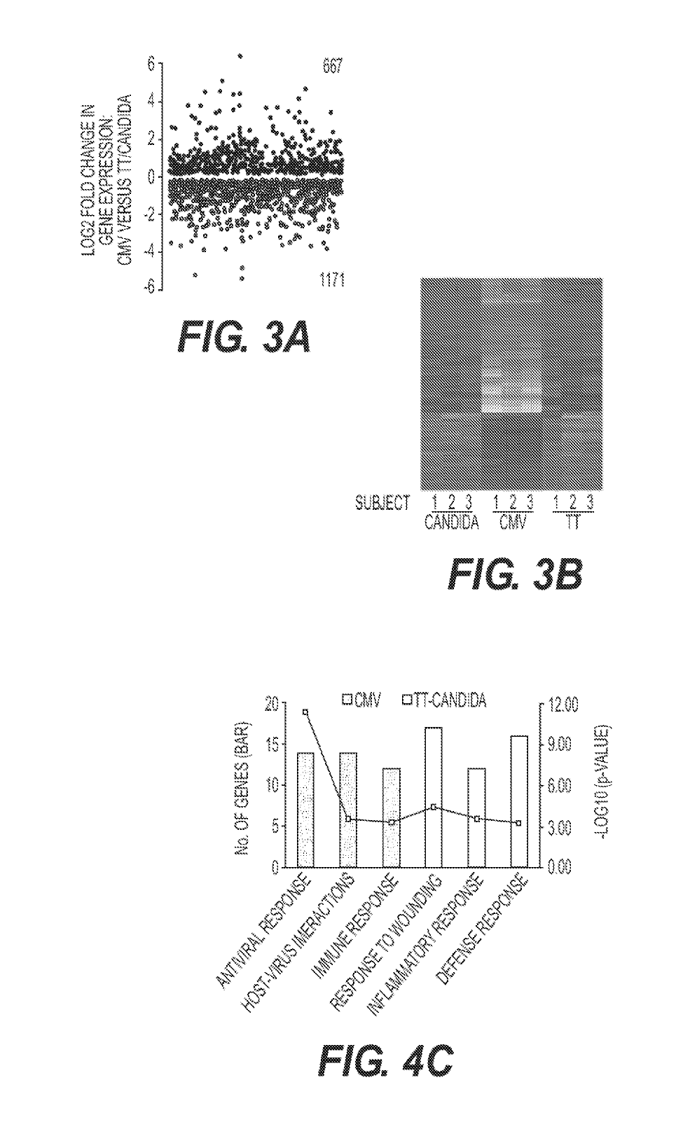

For co-receptors, CMV-specific CD4+ T cells expressed even higher surface CCR5 than TT- and Candida-specific CD4+ T cells (FIG. 3A). A similar expression pattern for CXCR4 was also observed. Neutralization of MIP-1α, but not MIP-1β or RANTES, led to substantial increase in intracellular p24 expression in TT- and Candida-specific CD4+ T cells (TT: 3.0% to 23.0%; Candida: 1.2% to 10.0%), and neutralizing all 3 β-chemokines had synergistic effect leading to the highest p24 expression (TT: 33.0%; Candida: 23.0%) (FIG. 3C middle and bottom).

These observations suggest that HIV infection of TT- and Candida-specific CD4+ T cells is largely restricted at entry, after which these 2 groups of pathogen-specific cells provide a permissive environment for HIV replication. In striking contrast, despite β-chemokine neutralization increased HIV full-length DNA in CMV-specific CD4+ T cells (FIG. 3B), p24 expression in these cells remained low (0.1% vs. 0.23%) (FIG. 3C top), suggesting that CMV-specific CD4+ T cells also restrict HIV replication at post-reverse transcription stages.

Despite higher surface expression, it is possible that the CCR5 on CMV-specific CD4+ T cells is less available to HIV for entry due to cellular factors, such as β-chemokines. Neutralization of MIP-1α, MIP-1β and RANTES substantially enhanced HIV full-length DNA in CD4+ T cells specific for the three pathogens compared to no neutralization (copy increase: 8.3 fold for CMV; 8.0 fold for TT; 11.8 fold for Candida) (FIG. 3B), which indicates functionality of the receptors for HIV entry on pathogen-specific CD4+ T cells.

To indentify the cellular factors that regulate the differential HIV infection between pathogen-specific CD4+ T cells, surface expression of CD4 and HIV co-receptors, CCR5 and CXCR4 were assessed. CD4 expression was comparable between pathogen-specific CD4+ T cells. Antigen-stimulated and HIV-infected PBMC were stained with aqua blue (Invitrogen) and antibody cocktails to surface antigens including CD4-ECD (Beckman Coulter), CD8-PE-Cy5, CD14-AF700, CD-19-AF700, CCR5-APC or CXCR4-APC (BD Bioscience). Antibody cocktail varied depending on different experiments. Cells were then fixed, permeabilized (BD Bioscience) and stained for CD3 (APC-H7; BD Bioscience) and p24 (PE; Beckman Coulter). Between 0.2 and 1×10⁶ cells were acquired by LSR-II (BD Bioscience). Antibody capture compensation beads (BD Bioscience) stained with individual antibodies were acquired for compensation. Data were analyzed using FlowJo (Tree Star, Inc.).

CFSE-labeled, antigen-stimulated PBMC were divided into two aliquots. One aliquot was infected with HIV, and 24 hours after infection cells were fixed and stained with aqua blue (Invitrogen) and antibody cocktail including anti-CD4-ECD (Beckman Coulter), anti-CD3-APC-H7, anti-CD8-PE-Cy5, anti-CD14/19-AF700 (BD Bioscience). The other aliquot was not HIV-infected and not subject to fixation. Live cells were stained with aqua blue and the same antibody cocktail as that for HIV-infected PBMC. CFSE-low CD3+CD8− T cells were sorted by FACS Aria (BD Bioscience).

Sorted, HIV-infected antigen-specific CD4+ T cells were subject to DNA extraction using crude cell lysis buffer (10 mM Tris-HCl, pH8; 1 mM EDTA; 0.001% Triton X 100; 0.001% SDS; with freshly added Proteinase K to 1 mg/ml). DNA quantification was performed using 2× TaqMan Universal PCR Master Mix and the 7500 Real Time PCR System (Applied Biosystems). Briefly, duplicate reactions for each sample were performed. Cycling parameters include: 95° C., 10 min; 50 cycles of 95° C., 15 sec, and 60° C., 1 min. Primers (Sigma-Aldrich) and probes (Sigma Aldrich and Applied Biosystems) sets included:
1) HIV-US1 strong stop: 5′R (US-1) [5′-AACTAGGGAACCCACTGCTTAA], 3′U5 [5′-TGAGGGATCTCTAGTTACCAGAGTCA], and R-probe [5′-(FAM)CCTCAATAAAGCTTGCCTTGAGTGCTTCAA(TAM)];
2) HIV-US1 full-length: 5′R(US-1), 3′gag [5′-CGAGTCCTGCGTCGAGAGA], and R-probe.

All amplifications were multiplexed with the GAPDH primer/probe set to both normalize sample input and serve as a DNA integrity control: GAPDH F [5′-ACCGGGAAGGAAATGAATGG], GAPDH R [5′-GCAGGAGCGCAGGGTTAGT], and GAPDH probe [5′ (VIC)ACCGGCAGGCTTTCCTAACGGCT(TAM)]. Final primer/probe concentrations were 100/200 nM, respectively; except for the GAPDH set: 75/100 nM. Normalized relative target expression was calculated as fold difference from cognate control values by 2^{(-ΔΔCt)}, where ΔΔCt=(ΔCt of the sample)−(ΔCt of the control); ΔCt=(average Ct of HIV target)−(average Ct of corresponding GAPDH).

Cellular RNA was extracted from sorted CFSE-low, antigen-specific CD4 populations using RNeasy Plus Mini Kit (Qiagen). RNA quality and concentration were assessed by Bioanalyzer (Agilent Technologies) and Nanodrop spectrophotometer (Thermo Scientific). Reverse transcription of total RNA and synthesis of biotin-labeled amplified RNA (aRNA) were performed using GeneChip IVT Express Kit (Affymetrix) according to the manufacturer's instructions. aRNA was fragmented and hybridized to GeneChip Human Genome-U133 plus 2.0 array (Affymetrix). The array was washed and stained with streptavidin phycoerythrin conjugate, followed by scanning on a GeneCHip Scanner. Data processing and analysis were performed using the R computing environment (available on the world-wide web at www.r-project.org/) version 2.12.2 with BioConductor packages (www.bioconductor.org). Gene expression data were normalized into Robust Multichip Average (RMA) expression measures and were compared between antigen specificities. Statistical analysis was performed using the Significance Analysis of Microarrays (SAM) 2.0. SAM scores were computed for each gene based on expression changes relative to the standard deviation. To control for multiple testing, false discovery rate (FDR) or the expected proportion of false positives among all significant genes identified was estimated based on SAM scores using 1,000 permutations. Genes with FDR below 0.05 were considered significant. Functional category and gene ontology enrichment analysis were performed using online tool DAVID based on significant genes identified by SAM.

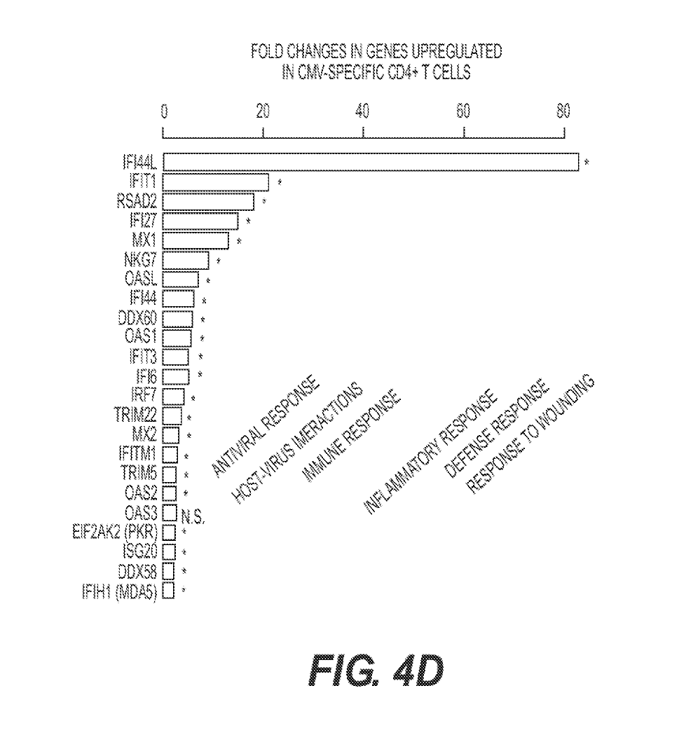

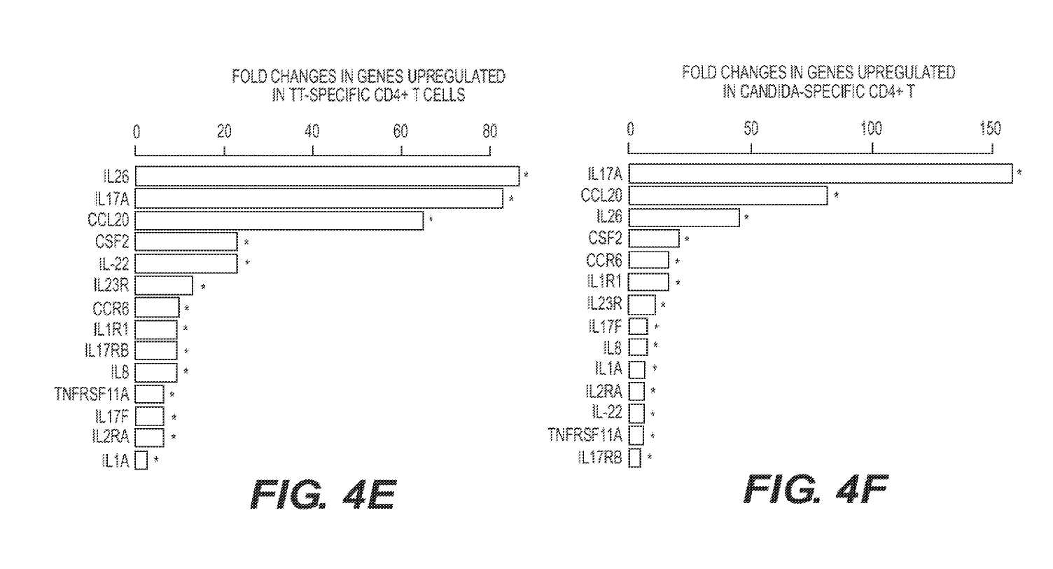

CFSE_{-low}, antigen-specific CD4+ T cells from the same donor PBMC were sorted and subjected to microarray analysis. There was a very distinct gene expression profile for CMV-specific CD4+ T cells compared to TT- and Candida-specific CD4+ T cells (FIGS. 4A and 4B). Functional category and gene ontology enrichment analysis identified that the profile of CMV-specific CD4+ T cells was dominated by responses linked to antiviral response and host-virus interactions, whereas the profiles of TT- and Candida-specific CD4+ T cells were mainly characterized by inflammatory and defense responses (FIG. 4C). For CMV-specific CD4+ T cells, comprehensive innate antiviral responses were activated, such as type-I IFN response (IFI44L, IFIT1, IFI27, IFI44, IFIT3, and IFI6, etc), antiviral RNA response (OASL, OAS1, OAS2, OAS3, PKR, MDA5), antiviral defense (RSAD2, MX1) and HIV/SIV restriction factors (TRIM22, TRIM5). A number of these genes were shown to have anti-immunodeficiency virus activity. IFI44L was the most upregulated with a more than 80-fold increase in gene expression. IFIT1 is an antiviral protein that recognizes 5′-triphosphate RNA and controls viral replication and was upregulated by more than 20-fold. This comprehensive antiviral profile provides an attractive explanation for the tropism-independent, multi-stage resistance to HIV of CMV-specific CD4+ T cells. Neither type-I IFN in CMV-stimulated PBMC nor preferential CMV infection of CMV-specific CD4+ T cells was detected. In contrast, the genes upregulated in TT- and Candida-specific CD4+ T cells were mainly associated with Th17 inflammatory response, such as IL-17A and IL-17F20. IL-17A expression was upregulated by more than 80-fold and 150-fold increases in TT- and Candida-specific cells, respectively. Other Th17 genes, IL-2221,22, IL-23R23 and IL-2624, and the genes induced by Th17 signaling, CCL2025 and its mucosal homing receptor CCR626, were also significantly up-regulated (FIGS. 4E and 4F).

This study support the notion that CMV-, TT- and Candida-specific CD4+ T cells differ markedly in susceptibility to HIV infection, with CMV-specific being least susceptible despite having higher CCR5 expression. This is associated with the selective upregulation of innate antiviral responses in CMV-specific cells. This suggests a mechanism for the preservation of CMV-specific responses and the earlier loss of Th17-associated TT- and Candida-specific responses seen in AIDS. The genes identified in these studies, such as IFI-44L32 and IFIT117, are useful in revealing new anti-HIV mechanisms and pathways.

1. A method for screening the resistance of CD4+ cells to viral infection, the method comprising
a) labeling isolated CD4+ cells with carboxyfluorescein diacetate, succinimidyl ester (CFSE),
b) contacting the labeled CD4+ cells with at least one composition to stimulate the CD4+ cells,
c) contacting the stimulated, labeled CD4+ cells with a subject virus, and
d) determining the infectivity of the subject virus in the stimulated, labeled CD4+ cells,
wherein a lower viral infectivity in the stimulated, labeled CD4+ cells compared to control cells indicates that the stimulated, labeled CD4+ cells are more resistant to infection of the virus than control cells.

2. The method of claim 1, wherein the subject virus is human immunodeficiency virus (HIV) and HIV infectivity is assessed in the stimulated, labeled CD4+ cells.

3. The method of claim 1, further comprising determining at least one gene that is transcribed at a higher level in the stimulated, labeled CD4+ cells exhibiting lower virus infectivity compared to control cells.

4. The method of claim 1, further comprising determining at least one gene that is transcribed at a lower level in the stimulated, labeled CD4+ cells exhibiting lower viral infectivity compared to control cells.

5. The method of claim 1, wherein determining the infectivity of the subject virus in the stimulated, labeled CD4+ cells comprises monitoring the expression of at least one marker indicative of infection of the subject virus.

6. The method of claim 5, wherein monitoring the expression of at least one marker comprises the use of fluorescence activated cell sorting (FACS).

7. The method of claim 1, wherein the composition comprises at least one antigen.

8. The method of claim 7, wherein the at least one antigen is or is derived from an animal pathogen.

9. The method of claim 8, wherein the pathogen is a virus, a bacterium, a prion or a fungus.

10. The method of claim 1, wherein the composition comprises at least one small molecule.

11. A method of inducing resistance of CD4+ cells to virus infection, the method comprising increasing the transcription of at least one gene selected from the group consisting of IFI44L, IFIT1, IFI27, IFI44, IFIT3, IFI6, OASL, OAS1, OAS2, OAS3, PKR, MDA5, RSAD2, MX1, TRIM22, TRIMS, NKG7, DDX60, IRF7, MX2, IFITM1, ISG20 and DDX58.

12. The method of claim 11, where in the virus is human immunodeficiency virus (HIV).

13. The method of claim 11, wherein increasing the transcription of at least one gene comprises transfecting CD4+ cells with at least one additional copy of the at least one gene.

14. The method of claim 11, wherein increasing the transcription of at least one gene comprises administering an agent to the CD4+ cells that causes transcription of the at least one gene.

15. A method of screening a composition for its ability to increase resistance of CD4+ cells to virus infection, the method comprising
a) contacting the composition with isolated CD4+ cells, and
b) assessing transcription levels of at least one gene selected from the group consisting of IFI44L, IFIT1, IFI27, IFI44, IFIT3, IFI6, OASL, OAS1, OAS2, OAS3, PKR, MDA5, RSAD2, MX1, TRIM22, TRIMS, NKG7, DDX60, IRF7, MX2, IFITM1, ISG20 and DDX58,
wherein an increase in transcription levels of the at least one gene compared to control levels indicates that the composition will increase the resistance of CD4+ cells to viral infection.

16. The method of claim 15, wherein the virus is human immunodeficiency virus (HIV).

17. The method of claim 15, wherein the composition is a vaccine or a potential vaccine.

18. The method of claim 15, wherein the transcription levels is assessed in a number of genes selected from the group consisting of 2, 3, 4, 5, 6, 7, 8, 9, 10, 11, 12, 13, 14, 15, 16, 17, 18, 19, 20, 21, 22 and 23.

19. The method of claim 15, wherein the composition comprises a small molecule.

20. The method of claim 15, wherein the composition comprises an antigen that is or is derived from an animal pathogen.


SEQUENCE LISTING (7 sequences)

SEQ ID NO: 1 - DNA, 22 bp, Artificial Sequence
  Primer/Probe
aactagggaacccactgcttaa

SEQ ID NO: 2 - DNA, 26 bp, Artificial Sequence
  Primer/Probe
tgagggatctctagttaccagagtca

SEQ ID NO: 3 - DNA, 30 bp, Artificial Sequence
  Primer/Probe
cctcaataaagcttgccttgagtgcttcaa

SEQ ID NO: 4 - DNA, 19 bp, Artificial Sequence
  Primer/Probe
cgagtcctgcgtcgagaga

SEQ ID NO: 5 - DNA, 20 bp, Artificial Sequence
  Primer/Probe
accgggaaggaaatgaatgg

SEQ ID NO: 6 - DNA, 19 bp, Artificial Sequence
  Primer/Probe
gcaggagcgcagggttagt

SEQ ID NO: 7 - DNA, 23 bp, Artificial Sequence
  Primer/Probe
accggcaggctttcctaacggct